(19) United States
(12) Patent Application Publication
(10) Pub. No.: US 2011/0166041 A1
(43) Pub. Date: Jul. 7, 2011

(54) Diagnosis/Therapeutic Strategy For Gynecological Cancer by Utilizing Micro-RNA as Biomarker

(75) Inventors: Takeru Zama, Tokyo (JP); Akira Hirasawa, Tokyo (JP); Koichiro Saito, Tokyo (JP); Tomoko Akahane, Tokyo (JP)

(73) Assignee: KEIO UNIVERSITY, Tokyo (JP)

(21) Appl. No.: 12/999,124

(22) PCT Filed: Jun. 25, 2009
(86) PCT No.: PCT/JP2009/002924

§ 371 Date: Feb. 28, 2011

(30) Foreign Application Priority Data
Jun. 27, 2008 (JP) 2008-168723
Dec. 5, 2008 (JP) 2008-310487

(51) Int. Cl. C40B 40/06; C12Q 1/68

(52) U.S. Cl. 506/16; 435/6.14; 435/6.11; 435/6.13

(57) ABSTRACT

Disclosed are: a method for using a particular microRNA as a biomarker for gynecological cancer; a method for the determination of gynecological cancer; a kit for the determination of gynecological cancer, and the like. The present invention is characterized in that at least one microRNA selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 is used as a biomarker for gynecological cancer.

### TECHNICAL FIELD

The present invention relates to an expression profile of a microRNA (miRNA) in gynecological cancer and a use thereof. More specifically, the present invention relates to a method using a specific microRNA as a biomarker for the gynecological cancer, a method for the determination of the gynecological cancer, a kit for the determination of the gynecological cancer, and the like.

### BACKGROUND ART

Gynecological cancer is a cancer occurring in the uterine cervix, uterine corpus, ovary, uterine tube, vagina, or external genitalia, and accounts for over ¼ the malignant tumor of women. The main gynecological cancers can include cervical cancer, endometrial cancer, and ovarian cancer (see Patent Document 1 and Non-Patent Document 1).

In recent years, basic and clinical studies on cancer have been dramatically advanced and demonstrated that cancer is a disease caused by abnormal genes. As a result, therapies are attempted which use various genes or proteins related to pathologic conditions of cancer as molecular targets. A cancer examination intended for early detection of uterine cancer has become widespread, and early medical treatment reduces mortality from cervical cancer. Meanwhile, however, the morbidity and mortality of endometrial cancer, ovarian cancer, and juvenile cervical cancer increase with increased female hormone stimulation due to a higher first-marriage age and a decrease in the number of pregnancies, changes in lifestyle such as a Westernized meal, or increased papilloma virus infection due to a tendency for younger people to be sexually active (see Non-Patent Document 2). Particularly in endometrial cancer, not only an increase in morbidity and mortality but also a tendency for women of lower age to suffer from the disease is observed (see Non-Patent Document 3).

In the diagnosis of gynecological cancer, various imaging tests (ultrasonography, CT, MRI, PET, etc.) are typically performed and the progress of the disease is further evaluated by an internal examination, with a metroscope, or by other means. Histopathological diagnosis is given on biopsy before surgery for cervical cancer and endometrial cancer, aside from ovarian cancer for which cytodiagnosis and histological diagnosis cannot be performed before surgery. Disease stage is determined on the basis of the progress and histopathological diagnosis of lesions. The disease stage of ovarian cancer cannot be determined until the histopathological diagnosis of the ovary isolated by abdominal operation is obtained. In addition, the progress of lesions determined by imaging tests is sometimes diverged from the pathological diagnosis thereof (see Non-Patent Document 4); thus, in the diagnosis of ovarian cancer, the possibility of malignancy must always be born in mind. Thus in the case of gynecological cancer, imaging tests cannot qualitatively diagnose the cancer and in many instances make it difficult to differentiate the cancer from benign disease. At present, the pathological diagnosis by cytodiagnosis and histological diagnosis, which is used as definite diagnosis, also sometimes makes the determination of progression and invasion degrees of the cancer difficult as well as not enabling the prediction of prognosis thereof even when the basic hematoxylin-eosin (HE) stain is used in combination with various immunostainings. Although several factors for prediction of onset and prognosis of the cancer are reported (see Patent Documents 4, 5 and 6), there currently exists no biomarker sufficient in both sensitivity and specificity.

Treatment policy for gynecological cancer is determined depending primarily on the disease stage and histopathological manifestation thereof. For benign tumor, radical single-stage extirpation or, if lesions are small and no clinical manifestation is observed, follow-up is carried out. On the other hand, for malignant tumor, surgery, chemotherapy with anticancer agents, and radiotherapy are carried out alone or in combination. For cervical cancer and endometrial cancer, it cannot be sometimes avoided that the uterine is removed by surgery; however, this poses serious problems because it causes the loss of fecundity in a young woman who has never borne a child (see Non-Patent Document 5). Ovarian cancer produces poor subjective symptoms and promptly becomes worse; thus, the cancer has often already made progress when it is detected. It is also difficult to early detect the cancer because an efficient screening method therefor has not been established (see Non-Patent Document 6) and ovarian cells cannot be harvested. In addition, even though it is early detected, in principle the uterine as an adjacent organ is subjected to complication ablation. Ovarian cancer occurs at all ages and causes the loss of fecundity particularly in the treatment of young individuals; thus, this poses serious problems as is the case with cervical cancer and endometrial cancer (see Non-Patent Document 7). For each cancer, chemotherapy is often post-operatively administered in the advanced type thereof; however, it has become a problem in that it has strong side effects such as reduced immune strength due to myelosuppression, dehydration due to digestive symptoms, and electrolyte imbalance. Further, individual variations are observed in therapeutic effect and prognosis and these are considered to be attributed to the mutation of genes and the abnormal expression level thereof in cancer tissue (see Non-Patent Document 8).

As described above, changes in lifestyle are predicted to increase gynecological cancer; thus, it is expected to develop biomarkers for prediction of onset and diagnosis of onset thereof. Ca19-9, Ca125, and the like are known as biomarkers used for the post-treatment follow-up of cervical cancer, but their positive rate is as low as about 30%; useful biomarkers for gynecological cancer have not heretofore been reported (see Patent Documents 2 and 3).

Meanwhile, a microRNA is a single-stranded RNA which is present in cells, not translated into protein, and on the order of 22 bases long (see Non-Patent Document 9). It was discovered in C. elegans in 1993 and thereafter in a vertebrate in 2001 and is conserved beyond species. Currently, about 1,000 microRNAs are predicted to be present on the human genome; 700 or more of microRNAs have so far been cloned. MicroRNAs are believed to control genes in 30% of protein-coding regions on the genome (see Non-Patent Document 10); therefore, the functional failure of microRNAs has a possibility of causing various diseases. However, to date a very few microRNAs have biological roles elucidated; future analysis is awaited. Reports of microRNAs for gynecological cancer are found here and there in the case of using cancer cell lines (see Non-Patent Documents 11 and 12); however, up to date only a few reports in which a patient's clinical specimen is used are found (see Non-Patent Document 13). Non-Patent Document 13 describes that the expression of mRNAs and microRNAs was compared between various stages and grades of endometrial cancer tissues and a normal endometrial tissue to identify 90 mRNAs and 13 microRNAs which were significantly different in the amount of expression. It is described that of the 13 microRNAs, 8 microRNAs, i.e., miR-185, miR-106a, miR-181a, miR-210, miR-423, miR-103, miR-107, and miR-let7c, increased in expression in endometrial cancer and 5 microRNAs, i.e., miR-let71, miR-221, miR-30c, miR-152, and miR-193, decreased in expression in endometrial cancer. However, Non-Patent Document 13 does not describe that the 13 microRNAs can be used for the diagnosis of endometrial cancer. The 13 microRNAs identified in Non-Patent Document 13 are also different from the present microRNAs.

### CITATION LIST

### Patent Document

Patent Document 1: National Publication of International Patent Application No. 2002-527723
Patent Document 2: National Publication of International Patent Application No. 2003-512010
Patent Document 3: Japanese Patent Laid-Open No. 2002-22746
Patent Document 4: Japanese Patent Laid-Open No. 2005-532314
Patent Document 5: U.S. Pat. No. 6,855,350
Patent Document 6: U.S. Pat. No. 7,045,292

### Non-Patent Document

Non-Patent Document 1: Whitcomb B P. Gynecologic Malignancies. Surg. Clin. North Am. 2008; 88: 301-317.
Non-Patent Document 2: Masatsugu Ueda Method for diagnosing cervical cancer—Recent topics—Jap. J. Med. Technol. 2003; 522: 179-186.
Non-Patent Document 3: Masatsugu Ueda Method for diagnosing endometrial cancer—Recent topics—Jap. J. Med. Technol. 2003; 52: 691-700.
Non-Patent Document 4: Ayhan A, Guven S, Guven E S, Kucukali T. Is there a correlation between tumor marker panel and tumor size and histopathology in well staged patients with borderline ovarian tumors? Acta Obstet. Gynecol. Scand. 2007; 86: 484-490.
Non-Patent Document 5: Amant F, Van Calsteren K, Vergote I, Ottevanger N. Gynecologic oncology in pregnancy. Crit. Rev. Oncol. Hematol. 2008. [Epub ahead of print]
Non-Patent Document 6: Jacobs I J, Menon U. Progress and challenges in screening for early detection of ovarian cancer. Mol. Cell. Proteomics. 2004; 3: 355-366.
Non-Patent Document 7: Chan J K, Tian C, Monk B J, Herzog T, Kapp D S, Bell J, Young R C; Gynecologic Oncology Group. Prognostic factors for high-risk early-stage epithelial ovarian cancer: a Gynecologic Oncology Group study. Cancer. 2008; 112: 2202-2010.
Non-Patent Document 8: Marsh S. Cancer pharmacogenetics. Methods Mol. Biol. 2008; 448: 437-446.
Non-Patent Document 9: Barbarotto E, Schmittgen T D, Calin G A. microRNAs and cancer: profile, profile, profile. Int. J. Cancer. 2008; 122(5): 969-977.
Non-Patent Document 10: Zeng Y, Yi R, Cullen B R. miRNAs and small interfering RNAs can inhibit mRNA expression by similar mechanisms. Proc. Nat. Acad. Sci. 2003; 100: 9779-9784.
Non-Patent Document 11: Jiang J, Lee E J, Gusev Y, Schmittgen TD. Real-time expression profiling of microRNA precursors in human cancer cell lines. Nucleic Acids Res. 2005; 28: 5394-5403.
Non-Patent Document 12: Tsuda N, Kawano K, Efferson C L, Ioannides C G. Synthetic microRNA and double-stranded RNA targeting the 3′-untranslated region of HER-2/neumRNA inhibit HER-2 protein expression in ovarian cancer cells. Int. J. Oncol. 2005; 27: 1299-1306.
Non-Patent Document 13: Boren T, Xiong Y, Hakam A, Wenham R, Apte S, Wei Z, Kamath S, Chen D T, Dressman H, Lancaster J M. MicroRNAs and their target messenger RNAs associated with endometrial carcinogenesis. Gynecol. Oncol. 2008. [Epub ahead of print]

### DISCLOSURE OF THE INVENTION

### Problems to be solved by the Invention

An object of the present invention is to provide a method using a specific microRNA as a biomarker for gynecological cancer, a method for the determination of gynecological cancer, a kit for the determination of gynecological cancer, and the like.

### Means for Solving the Problems

As described above, Non-Patent Document 13 discloses the 13 microRNAs suggested to be related to endometrial cancer. However, the 13 microRNAs identified in Non-Patent Document 13 are different from the present microRNAs, and Non-Patent Document 13 does not describe that the 13 microRNAs can be used for the diagnosis of endometrial cancer. In addition, the number of microRNAs which were the object of analysis in the Non-Patent Document was as insufficient as 335. Under such circumstances, the present inventors have compared 723 microRNAs between endometrial cancer tissue and uterine polyp (uterine benign tumor) tissue and have found that particular microRNAs are abnormally expressed in the endometrial cancer tissue, thereby accomplishing the present invention.

Specifically, the present invention relates to (1) a method for using one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 as a biomarker for gynecological cancer and (2) the method according to item (1) above, wherein the miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 are RNAs in which one or more nucleotides are deleted, substituted or added in the nucleotide sequences represented by SEQ ID NOS: 1 to 27 and 50 to 63, respectively and whose expression increases or decreases in a gynecological cancer tissue or blood of a gynecological cancer subject compared to a control.

The present invention also relates to (3) a method for the determination of gynecological cancer, comprising the steps of: (A) extracting RNA from a gynecological specimen tissue or blood of a subject; (B) measuring the expression level of one or more microRNAs selected from miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 in the extracted RNA; and (C) comparing the measured expression level to the expression level of the microRNAs in a non-cancer tissue of the same type as that of the specimen tissue or in blood of a normal subject of the same species as that of the above subject as a control for evaluation, (4) the method for the determination of gynecological cancer according to item (3) above, wherein the miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 are RNAs in which one or more nucleotides are deleted, substituted or added in the nucleotide sequences represented by SEQ ID NOS: 1 to 27 and 50 to 63, respectively and whose expression increases or decreases in the gynecological cancer tissue or the blood of the gynecological cancer subject compared to a control, (5) the method for the determination of gynecological cancer according to item (3) or (4) above, wherein the cancer is determined as being gynecological cancer when the expression level of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-138, miR-203, miR-488, miR-489, miR-643, miR-885-5p, and miR-888 in the extracted RNA increases compared to a control, and (6) the method for the determination of gynecological cancer according to item (3) or (4) above, wherein the cancer is determined as being gynecological cancer when the expression level of one or more microRNAs selected from the group of microRNAs consisting of miR-377*, miR-449b, miR-449a, miR-369-3p, miR-323-3p, miR-329, miR-299-5p, miR-34b, miR-411, miR-34c-5p, miR-376b, miR-337-3p, miR-337-5p, miR-127-3p, miR-424, miR-432, miR-433, miR-450a, miR-503, miR-542-3p, miR-542-5p and miR-584 in the extracted RNA decreases compared to a control.

The present invention further relates to (7) a kit for the determination of gynecological cancer, comprising a microarray which includes a polynucleotide consisting of a nucleic acid sequence complementary to the sequence of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 or a part thereof, and which is capable of measuring the expression level of the microRNA, (8) a kit for the determination of gynecological cancer, including a primer set capable of amplifying the sequence of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 and a fluorescent probe comprising a polynucleotide consisting of a nucleic acid sequence complementary to the microRNA sequence or a part thereof, and (9) the determination kit according to item (7) or (8) above, wherein the miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 are RNAs in which one or more nucleotides are deleted, substituted or added in the nucleotide sequences represented by SEQ ID NOS: 1 to 27 and 50 to 63, respectively and whose expression increases or decreases in a gynecological cancer tissue or blood of a gynecological cancer subject compared to a control.

The present invention still further relates to (10) a method for screening a therapeutic agent for gynecological cancer, comprising the steps of: (A) administering a test substance to a non-human animal suffering from gynecological cancer; (B) measuring the expression level of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 in a gynecological cancer tissue or blood of the non-human animal; and (C) comparing the above expression level to the expression level of the microRNA in the case of the test substance being unadministered as a control for evaluation, and (11) the screening method according to item (10) above, wherein the miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 are RNAs in which one or more nucleotides are deleted, substituted or added in the nucleotide sequences represented by SEQ ID NOS: 1 to 27 and 50 to 63, respectively and whose expression increases or decreases in the gynecological cancer tissue or the blood of the gynecological cancer subject compared to a control.

### Advantages of the Invention

The method for the determination of gynecological cancer and the method for using a microRNA as a biomarker for gynecological cancer according to the present invention enable the gynecological cancer to be rapidly and accurately determined. The kit for the determination of gynecological cancer and the method for using a microRNA for the determination of gynecological cancer according to the present invention also enable the rapid and accurate determination of the presence of the gynecological cancer. In addition, the method for screening therapeutic agents for gynecological cancer enables the therapeutic agents for gynecological cancer to be efficiently screened. Particularly, a microRNA whose concentration in the blood of a subject also changes when a subject suffers from gynecological cancer is excellent especially in terms of speeding-up and simplifying the method for use as a biomarker for gynecological cancer, the method for the determination of gynecological cancer, and the like because a blood sample very easy to collect can be used. Further advances in research on the present microRNAs enables the prediction of onset, progress and prognosis and treatment of gynecological cancer, which can be expected to offer a breakthrough toward the solution of various current clinical problems with the gynecological cancer. The expression of a microRNA is known to be tumor tissue-specific and also has a possibility of being useful for identification of the primary site of gynecological cancer of unknown primary origin. In the treatment of the cancer, if resistance to drug and radiation therapies can be diagnosed, it further has a possibility of being developed up to therapy for individual in which an effective therapy is selected for each individual patient. Accurate diagnosis and prediction of prognosis can be expected to reduce excessive tests and extra hospital visits, which is also expected to have a great medical economic effect.

### BRIEF DESCRIPTION OF DRAWINGS

FIG. 1 A drawing summarizing samples for analyzing the expression profile of microRNAs.

FIG. 2 A drawing showing the results of quality checking of sample-derived total RNA using UV.

FIG. 3 A series of graphs showing the results of quality checking of sample-derived total RNA using Bio-Analyzer RNA6000.

FIG. 4 A series of graphs showing the relative expression level of microRNAs (miR-592, miR-629*, miR-517a, miR-205, miR-184, miR-509-5p, miR-135a*, miR-137, miR-602, miR-186*, miR-181a-2*, and miR-193b*) whose expression increases in an endometrial cancer tissue in each sample. Bar graphs in each panel represent the relative expression level thereof in samples: polyp, cancer (1), cancer (2) and cancer (3), respectively from the left.

FIG. 5 A series of graphs showing the relative expression level of microRNAs (miR-377*, miR-449b, miR-449a, miR-369-3p, miR-323-3p, miR-329, miR-299-5p, miR-34b, miR-411, miR-34c-5p, miR-376b, miR-337-3p, miR-337-5p, and miR-127-3p) whose expression decreases in an endometrial cancer tissue in each sample. Bar graphs in each panel represent the relative expression level thereof in samples: polyp, cancer (1), cancer (2) and cancer (3), respectively from the left.

FIG. 6 A series of graphs showing the relative expression level of members of the let-7 family in a non-cancer tissue or cancer tissues of the uterine corpus. Bar graphs in each panel represent the relative expression level thereof in samples: polyp, cancer (1), cancer (2) and cancer (3), respectively from the left.

FIG. 7 A series of graphs showing the relative expression level of members of the miR-17-92 cluster in a non-cancer tissue or cancer tissues of the uterine corpus. Bar graphs in each panel represent the relative expression level thereof in samples: polyp, cancer (1), cancer (2) and cancer (3), respectively from the left.

FIG. 8 A series of graphs showing the relative expression level of miR-15 and miR-16 in a non-cancer tissue or cancer tissues of the uterine corpus. Bar graphs in each panel represent the relative expression level thereof in samples: polyp, cancer (1), cancer (2) and cancer (3), respectively from the left.

FIG. 9 A drawing summarizing samples for analyzing the expression profile of microRNAs.

FIG. 10 A drawing showing the results of quality checking of sample-derived total RNA using UV and Bio-Analyzer RNA6000.

FIG. 11 A series of graphs showing the relative expression level of microRNAs (miR-135a*, miR-137, miR-138, miR-203, miR-205, and miR-488) whose expression increases in an endometrial cancer tissue in each sample. Bar graphs in each panel represent the relative expression level thereof in samples: “42 (NORMAL)” (normal endometrial tissue), “06-072 (G1Ib)”, “05-034 (G1Ib)”, “04-010 (G33IIIc)”, and “06-082 (G3IIIc)”, respectively from the left.

FIG. 12 A series of graphs showing the relative expression level of microRNAs (miR-489, miR-629*, miR-643, miR-885-5p, and miR-888) whose expression increases in an endometrial cancer tissue in each sample. Bar graphs in each panel represent the relative expression level thereof in samples: “42 (NORMAL)” (normal endometrial tissue), “06-072 (G1Ib)”, “05-034 (G1Ib)”, “04-010 (G33IIIc)”, and “06-082 (G3IIIc)”, respectively from the left.

FIG. 13 A series of graphs showing the relative expression level of microRNAs (miR-34c-5p, miR-127-3p, miR-299-5p, miR-323-3p, miR-337-5p, miR-376b, miR-411, miR-424, and miR-432) whose expression decreases in an endometrial cancer tissue in each sample. Bar graphs in each panel represent the relative expression level thereof in samples: “42 (NORMAL)” (normal endometrial tissue), “06-072 (G1Ib)”, “05-034 (G1Ib)”, “04-010 (G33IIIc)”, and “06-082 (G3IIIc)”, respectively from the left.

FIG. 14 A series of graphs showing the relative expression level of microRNAs (miR-433, miR-450a, miR-503, miR-542-3p, miR-542-5p and miR-584) whose expression decreases in an endometrial cancer tissue in each sample. Bar graphs in each panel represent the relative expression level thereof in samples: “42 (NORMAL)” (normal endometrial tissue), “06-072 (G1Ib)”, “05-034 (G1Ib)”, “04-010 (G33IIIc)”, and “06-082 (G3IIIc)”, respectively from the left.

### BEST MODE OF CARRYING OUT THE INVENTION

The method for using a microRNA as a biomarker for gynecological cancer according to the present invention is not particularly limited provided that it is a method for using one or more microRNAs selected from the group of microRNAs consisting of miR-592 (microRNA-592), miR-629* (microRNA-629*), miR-517a (microRNA-517a), miR-205 (microRNA-205), miR-184 (microRNA-184), miR-509-5p (microRNA-509-5p), miR-135a* (microRNA-135a*), miR-137 (microRNA-137), miR-602 (microRNA-602), miR-186* (microRNA-186*), miR-181a-2* (microRNA-181a-2*), miR-193b* (microRNA-193b*), miR-377* (microRNA-377*), miR-449b (microRNA-449b), miR-449a (microRNA-449a), miR-369-3p (microRNA-369-3p), miR-323-3p (microRNA-323-3p), miR-329 (microRNA-329), miR-299-5p (microRNA-299-5p), miR-34b (microRNA-34b), miR-411 (microRNA-411), miR-34c-5p (microRNA-34c-5p), miR-376b (microRNA-376b), miR-885-5p (microRNA-885-5p), miR-337-3p (microRNA-337-3p), miR-337-5p (microRNA-337-5p), miR-127-3p (microRNA-127-3p), miR-138 (microRNA-138, miR-203 (microRNA-203), miR-488 (microRNA-488), miR-489 (microRNA-489), miR-643 (microRNA-643), miR-888 (microRNA-888), miR-424 (microRNA-424), miR-432 (microRNA-432), miR-433 (microRNA-433), miR-450a (microRNA-450a), miR-503 (microRNA-503), miR-542-3p (microRNA-542-3p), miR-542-5p (microRNA-542-5p) and miR-584 (microRNA-584) (hereinafter also referred to as “the present microRNAs”) as biomarkers for gynecological cancers such as endometrial cancer; specific examples thereof can include the method for the determination of gynecological cancer according to the present invention, the method for use for the determination of gynecological cancer according to the present invention, and the method for screening therapeutic agents for gynecological cancer according to the present invention.

The gynecological cancer means a cancer occurring in the uterine cervix, uterine corpus, ovary, vagina, or external genitalia. Among these gynecological cancers, cancers of uterine cervix and uterine corpus can be preferably exemplified; among others, endometrial cancer can be more preferably exemplified. The type of the above cancers is not particularly limited to incipient or recurrent cancer; however, incipient cancer can be more preferably exemplified.

Preferred examples of living organisms from which the present microRNAs are derived can include mammals such as a human, a mouse, a rat, a hamster, a guinea pig, a monkey, a cow, a pig, a horse, a rabbit, sheep, a goat, a cat, and a dog; among others, a human and a mouse can be more preferably exemplified; and a human can be particularly preferably exemplified. The present microRNAs of human origin miR-592 (SEQ ID NO:1), miR-629* (SEQ ID NO:2), miR-517a (SEQ ID NO:3), miR-205 (SEQ ID NO:4), miR-184 (SEQ ID NO:5), miR-509-5p (SEQ ID NO:6), miR-135a* (SEQ ID NO:7), miR-137 (SEQ ID NO:8), miR-602 (SEQ ID NO:9), miR-186* (SEQ ID NO:10), miR-181a-2* (SEQ ID NO:11), miR-193b* (SEQ ID NO:12), miR-377* (SEQ ID NO:13), miR-449b (SEQ ID NO:14), miR-449a (SEQ ID NO:15), miR-369-3p (SEQ ID NO:16), miR-323-3p (SEQ ID NO:17), miR-329 (SEQ ID NO:18), miR-299-5p (SEQ ID NO:19), miR-34b (SEQ ID NO:20), miR-411 (SEQ ID NO:21), miR-34c-5p (SEQ ID NO:22), miR-376b (SEQ ID NO:23), miR-885-5p (SEQ ID NO:24), miR-337-3p (SEQ ID NO:25), miR-337-5p (SEQ ID NO:26), miR-127-3p (SEQ ID NO:27), miR-138 (SEQ ID NO:50), miR-203 (SEQ ID NO:51), miR-488 (SEQ ID NO:52), miR-489 (SEQ ID NO:53), miR-643 (SEQ ID NO:54), miR-888 (SEQ ID NO 55), miR-424 (SEQ ID NO 56), miR-432 (SEQ ID NO:57), miR-433 (SEQ ID NO:58), miR-450a (SEQ ID NO:59), miR-503 (SEQ ID NO:60), miR-542-3p (SEQ ID NO:61), miR-542-5p (SEQ ID NO:62) and miR-584 (SEQ ID NO:63) have nucleotide sequences as represented by SEQ ID NOS: 1 to 27 and 50 to 63, respectively. The sequence of microRNA is particularly high in conservativeness among mammals; the present microRNA even in mammals other than a human probably has an expression profile similar to that for a human. For example, the present microRNAs of mouse origin miR-592 (SEQ ID NO:45), miR-449b (SEQ ID NO:46), miR-369-3p (SEQ ID NO:47), miR-329 (SEQ ID NO:48), and miR-411 (SEQ ID NO:49) have nucleotide sequences as represented by SEQ ID NOS: 45 to 49, respectively.

For convenience the present microRNA includes RNA in which one or more nucleotides are deleted, substituted or added in the nucleotide sequences represented by SEQ ID NOS: 1 to 27 and 50 to 63 and whose expression increases or decreases in a gynecological cancer tissue or blood of a gynecological cancer subject compared to a control. The above “at least one” is preferably 1 to 5, more preferably 1 to 3, still more preferably 1. It can be easily confirmed, for example, by a microarray or quantitative PCR method to be described later whether the RNA consisting of a nucleotide sequence having deletion or the like increases or decreases in expression in the gynecological cancer tissue or blood of a gynecological cancer subject compared to a control. The present microRNAs from the above living organisms other than a human can be confirmed or identified based on the information deposited in databases such as GenBank.

The method for the determination of gynecological cancer according to the present invention is not particularly limited provided that it comprises the steps of: (A) extracting RNA from gynecological specimen tissue or blood of a subject; (B) measuring the amount of the present microRNAs in the extracted RNA; and (C) comparing the measured expression level with the expression level of the microRNAs in a non-cancer tissue of the same type as that of the specimen tissue or in blood of a normal subject of the same species as that of the above subject as a control for evaluation; however, preferred examples thereof can include a method in which the cancer is determined as being gynecological cancer such as endometrial cancer when the expression level of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-138, miR-203, miR-488, miR-489, miR-643, miR-885-5p, and miR-888 in the extracted RNA increases compared to a control and/or determined as being gynecological cancer such as endometrial cancer when the expression level of one or more microRNAs selected from the group of microRNAs consisting of miR-377*, miR-449b, miR-449a, miR-369-3p, miR-323-3p, miR-329, miR-299-5p, miR-34b, miR-411, miR-34c-5p, miR-376b, miR-337-3p, miR-337-5p, miR-127-3p, miR-424, miR-432, miR-433, miR-450a, miR-503, miR-542-3p, miR-542-5p and miR-584 in the extracted RNA decreases compared to a control. When the gynecological specimen tissue is used in the above step (A), a normal tissue of the same type as that of the specimen tissue is used as a control in the above step (C); and when the blood of the subject is used in the step (A), blood of a normal subject of the same species as that of the above subject is used as a control in the step (C). The expression level of a microRNA herein preferably uses the relative expression level corrected using a suitable internal standard gene to correct variations between samples in view of performing accurate determination. The internal standard gene is not particularly limited; examples thereof can include at least one gene selected from the group consisting of RNU48, RNU6b, snRNA (small nuclear RNA), and snoRNA (small nucleolar RNA).

The method for extracting RNA in the above (A) step is not particularly limited provided that it is a method which involves extracting RNA comprising microRNAs from the gynecological specimen tissue or the blood of the subject; preferred examples thereof can include a method which involves extracting total RNA comprising microRNAs using mirVana miRNA Isolation Kit (from Applied Biosystems) according to the appended protocol. The gynecological specimen tissue is not particularly limited provided that it is a tissue selected from uterine cervix, uterine corpus, ovary, vagina, and external genitalia, derived from a living organism whose gynecological cancer is to be determined. Preferred examples of the living organism can include mammals such as a human, a mouse, a rat, a hamster, a guinea pig, a monkey, a cow, a pig, a horse, a rabbit, sheep, a goat, a cat, and a dog; among others, a human and a mouse can be preferably exemplified; and a human can be particularly preferably exemplified.

The method for measuring the expression level of the present microRNA in the above (B) step is not particularly limited provided that it is a method which can identify the amount of the present microRNA in RNA comprising microRNAs; preferred examples thereof can include: a method for using the kit for the determination of gynecological cancers such as endometrial cancer according to the present invention, that is, the kit for the determination of gynecological cancer comprising a microarray including a polynucleotide consisting of a nucleic acid sequence complementary to the sequence of the present microRNA or a part thereof, capable of measuring the expression level of the present microRNA, the kit for the determination of gynecological cancer including a primer set capable of amplifying the sequence of the present microRNA, or the kit for the determination of gynecological cancer including a primer set capable of amplifying the sequence of the present microRNA and a fluorescent probe comprising a polynucleotide consisting of a nucleic acid sequence complementary to the sequence of the present microRNA or a part thereof; and the method for use for the determination of gynecological cancers such as endometrial cancer according to the present invention, that is, the microarray method for using for the determination of gynecological cancer in which a microarray includes a polynucleotide consisting of a nucleic acid sequence complementary to the sequence of the present microRNA or a part thereof, capable of measuring the expression level of the present microRNA, the quantitative PCR method using a primer set capable of amplifying the sequence of the present microRNA for the determination of gynecological cancer, or the quantitative PCR method (fluorescent probe method) for the determination of gynecological cancer using a primer set capable of amplifying the sequence of the present microRNA and a fluorescent probe comprising a polynucleotide consisting of a nucleic acid sequence complementary to the sequence of the present microRNA or a part thereof.

The microarray method is not particularly limited provided that it can measure the expression level of the present microRNA; examples thereof can include a method which involves labeling the RNA extracted from a tissue with a label (preferably a fluorescent label), contacting the RNA with a microarray to which a probe consisting of a polynucleotide (preferably DNA) consisting of a nucleic acid sequence complementary to the microRNA to be identified or a part thereof is fixed for hybridization, washing the microarray, and measuring the expression level of the remaining microRNAs on the microarray.

The type of the nucleotide of the nucleic acid sequence is not particularly limited provided that it can specifically hybridize to the predetermined microRNA according to the present invention; however, preferred is DNA because of its high stability. The length of the part of the polynucleotide is not particularly limited provided that it specifically hybridizes to the predetermined microRNA according to the present invention; however, it is preferably 10 to 100 mers, more preferably 10 to 40 mers in view of securing the stability of hybridization. The polynucleotide or a part thereof can be obtained by chemical synthesis or the like using a method well known in the art.

The array to which the polynucleotide or a part thereof is fixed is not particularly limited; however, preferred examples thereof can include a glass substrate and a silicon substrate, and the glass substrate can be preferably exemplified. A method for fixing the polynucleotide or a part thereof to the array is not particularly limited; a well-known method may be used.

The kit for the determination of gynecological cancer comprising a microarray according to the present invention may further comprise any element, such as reagents used for a microarray method, including, for example, a reagent used for RNA-labeling reaction, a reagent used for hybridization, a reagent used for washing, and a reagent used for extracting RNA from a tissue in addition to the above-described microarray.

The microarray method can be specifically exemplified by a method which involves measuring the expression level of a microRNA on DNA Microarray Scanner (from Agilent Technologies) using Agilent Human miRNA V2 (from Agilent Technologies) according to the method described in Agilent Technologies' miRNA Microarray Protocol Version 1.5. The microarray to which the probe consisting of the polynucleotide or a part thereof is fixed can be prepared, for example, by synthesizing a polynucleotide based on the sequence information of the present microRNA to be measured and fixing it to a commercially available array.

The quantitative PCR method is not particularly limited provided that it is a method using a primer set capable of amplifying the sequence of the present microRNA and can measure the expression level of the present microRNA; conventional quantitative PCR methods such as an agarose electrophoresis method, an SYBR green method, and a fluorescent probe method may be used. However, the fluorescent probe method is most preferable in terms of the accuracy and reliability of quantitative determination.

The primer set for the quantitative PCR method means a combination of primers (polynucleotides) capable of amplifying the sequence of the present microRNA. The primers are not particularly limited provided that they can amplify the sequence of the present microRNA; examples thereof can include a primer set consisting of a primer consisting of the sequence of a 5′ portion of the sequence of a predetermined microRNA of the present invention (forward primer) and a primer consisting of a sequence complementary to the sequence of a 3′ portion of the microRNA (reverse primer). Here, the 5′ means 5′ to the sequence corresponding to the reverse primer when both primers were positionally compared in the sequence of a mature microRNA; the 3′ means 3′ to the sequence corresponding to the forward primer when both primers were positionally compared in the sequence of a mature microRNA.

Preferred examples of the 5′ sequence of a microRNA can include a sequence 5′ to the central nucleic acid of the microRNA sequence; preferred examples of the 3′ sequence of the microRNA can include a sequence 3′ to the central nucleic acid of the microRNA sequence. The length of each primer is not particularly limited provided that it enables the amplification of the microRNA; however, each primer is preferably a 7-to-10-mer polynucleotide. The type of the nucleotide of a polynucleotide as the primer is preferably DNA because of its high stability.

The fluorescent probe is not particularly limited provided that it comprises a polynucleotide consisting of a nucleic acid sequence complementary to the sequence of the present microRNA or a part thereof; preferred examples thereof can include a fluorescent probe capable of being used for the TaqMan (trade name) probe method (including one using the FRET principle) or the cycling probe method; the fluorescent probe capable of being used for the TaqMan (trade name) probe method can be particularly preferably exemplified. Examples of the fluorescent probe capable of being used for the TaqMan (trade name) probe method (excluding one using the FRET principle) or the cycling probe method can include a fluorescent probe in which a fluorochrome is labeled 5′ thereof and a quencher is labeled on 3′ thereof. Examples of the fluorescent probe using the FRET principle can include a fluorescent probe in which a donor dye and an acceptor dye are labeled 5′ and 3′ thereof, respectively. The fluorochrome, quencher, donor dye, acceptor dye or the like used may be a commercially available one as needed.

The type of the nucleotide of the nucleic acid sequence in the fluorescent probe is not particularly limited provided that it can specifically hybridize to the present microRNA; however, it is preferably DNA because of its high stability. The length of the part of the polynucleotide is not particularly limited provided that it specifically hybridize to the predetermined microRNA according to the present invention; however, it is preferably 10 mers or more, more preferably 15 mers or more in view of securing the stability of hybridization and the part still more preferably has a number of nucleotides (1 to 3 mers) smaller than the number of nucleotides of a full-length microRNA of interest.

The primer set and the fluorescent probe can be obtained based on their sequence information by chemical synthesis or the like using a method well known in the art. Preferred specific methods for quantitative PCR using the primer set and fluorescent probe can include a method which involves using TaqMan (trade name) microRNA Assays (from Applied Biosystems) according to the appended protocol. The kit for the determination of gynecological cancer including a primer set and a fluorescent probe may further comprise any element such as, for example, a reagent used for quantitative PCR reaction such as polymerase in addition to the above-described primer set.

The non-cancer tissue of the same type as that of the specimen tissue (for example, a gynecological non-cancer tissue when the specimen tissue is a gynecological specimen tissue) or the blood of a normal subject of the same species as that of the subject (for example, the blood of a human as a non-gynecological cancer patient when a subject is the human) as a control in the (C) step is preferably a non-cancer tissue (non-cancer region) of the same individual as that from which the specimen tissue is derived because it enables more accurate evaluation in the method involving comparison with the expression level of a microRNA in the normal tissue for evaluation. The normal tissue of the same type as that of the specimen tissue or the blood of the normal subject of the same species as that of the subject as a control does not necessarily need to be collected in collecting the specimen tissue or the blood of the subject; the comparison may be performed with a calibration curve prepared in advance for the expression level of the present microRNA in the non-cancer tissue or the blood of the normal subject for evaluation. Preferred examples of the non-cancer tissue can include benign tumor tissues such as polyp and normal tissues.

In this manner, based on the expression level of microRNAs contained in RNA in a specimen tissue or the blood of a subject, it can be determined whether the specimen tissue or the subject has gynecological cancer or not (whether the specimen tissue has gynecological cancer or whether the subject suffers from gynecological cancer). As described above, an increase in the amount of miR-592, miR-629*, miR-517a, miR-205, miR-184, miR-509-5p, miR-135a*, miR-137, miR-602, miR-186*, miR-181a-2*, miR-193b*, miR-138, miR-203, miR-488, miR-489, miR-643, miR-885-5p, and miR-888 among the present microRNAs compared to a control can be determined as indicating gynecological cancer; a decrease in the amount of miR-377*, miR-449b, miR-449a, miR-369-3p, miR-323-3p, miR-329, miR-299-5p, miR-34b, miR-411, miR-34c-5p, miR-376b, miR-337-3p, miR-337-5p, miR-127-3p, miR-424, miR-432, miR-433, miR-450a, miR-503, miR-542-3p, miR-542-5p and miR-584 thereamong compared to a control can be determined as indicating gynecological cancer.

Among the present microRNAs, miR-137, miR-138, miR-203, miR-205, miR-488, miR-489, miR-643, miR-885-5p, and miR-888 have average expression levels in cancer tissue of 5 times or more of those in non-cancer tissue, showing a large difference in the amount of expression therebetween; thus, they are preferable in that they enable the more accurate determination of the presence of gynecological cancer. The measurement of the expression level of the present two or more microRNAs to compare with a control for evaluation is also preferable in that it enables the more accurate determination of the presence of gynecological cancer such as endometrial cancer. In PCT/JP2009/002629 which is a PCT application for a distinct invention of the present inventors (diagnosis/treatment option for head-and-neck tumor using microRNA as biomarker), microRNAs (e.g., miR-196a) whose concentration in the blood of a subject is changed when the subject suffers from head-and-neck tumor have actually been found. There is probably also an extremely high possibility of the presence of microRNAs whose concentration in the blood of a subject changes when the subject suffers from gynecological cancer, among the present microRNAs whose expression increases or decreases in gynecological cancer tissue. Among the present microRNAs, microRNAs whose concentration in the blood of a subject changes when the subject suffers from gynecological cancer are also very preferable in that they make the method for using them as a biomarker for gynecological cancer, the method for the determination of gynecological cancer, and the like particularly excellent in rapidity and simplicity since a blood sample, extremely easy to collect, can be utilized. Which of the present microRNAs is changed in the concentration thereof in the blood of a subject when the subject suffers from gynecological cancer can be easily found by comparing the concentration of the present microRNAs in the blood of the subject suffering from gynecological cancer with the concentration of the present microRNAs in the blood of a normal subject not suffering from gynecological cancer.

The degree of an increase in the expression level of the present microRNA when determined as indicating gynecological cancer can be, for example, preferably 100% or more, more preferably 150% or more, still more preferably 200% or more as a percentage relative to a control, and the degree of a decrease in the expression level of the present microRNA when determined as indicating gynecological cancer can be, for example, preferably 50% or more, more preferably 75% or more, still more preferably 90% or more as a percentage relative to a control.

In addition to the above steps (A) to (C), the method for the determination of gynecological cancer according to the present invention may further comprise the step of (D) measuring the expression level of one or more microRNAs selected from the group of microRNAs consisting of let-7 family, miR-17-92 cluster, miR-15, and miR-16 in the extracted RNA and comparing the expression level of the microRNA in the non-cancer tissue of the same type as that of the specimen tissue as a control for evaluation. Preferred examples of the member of the let-7 family can include let-7a, let-7b, let-7c, let-7d, let-7e, let-7f, let-7g, and let-7i. Preferred examples of the member of the miR-17-92 cluster can include miR-17, miR-18a, miR-19a, miR-19b, miR-20a, and miR-92a. Preferred examples of the miR-15 can include miR-15a and miR-15b. Particularly preferred examples of the microRNAs (let-7a (SEQ ID NO:28), let-7b (SEQ ID NO:29), let-7c (SEQ ID NO:30), let-7d (SEQ ID NO:31), let-7e (SEQ ID NO:32), let-7f (SEQ ID NO:33), let-7g (SEQ ID NO:34), let-71 (SEQ ID NO:35), miR-17 (SEQ ID NO:36), miR-18a (SEQ ID NO:37), miR-19a (SEQ ID NO:38), miR-19b (SEQ ID NO:39), miR-20a (SEQ ID NO:40), miR-92a (SEQ ID NO:41), miR-15a (SEQ ID NO:42), miR-15b (SEQ ID NO:43), and miR-16 (SEQ ID NO:44)) derived from humans can include RNAs consisting of nucleotide sequences represented by SEQ ID NOS: 28 to 44, respectively.

Each of the microRNAs (let-7family, miR-17-92 cluster, miR-15, and miR-16) in the step (D) shows abnormal expression in cancers other than gynecological cancer; thus, when the specimen tissue is determined to have gynecological cancer as a result of identification and evaluation in the step (B), the further observation of abnormal expression of such a microRNA can result in the determination that lesions of cancers other than gynecological cancer are present. In fact, the expression of the microRNA let-7 family or miR-15 and miR-16 is known to decrease in lung cancer tissue or chronic lymphatic leukemia or pancreas cancer tissue, respectively, and the expression of miR-17-92 cluster is known to increase in B cell lymphoma or lung cancer tissue.

The method for screening therapeutic agents for gynecological cancer according to the present invention is not particularly limited provided that it comprises the steps of: (a) administering a test substance to a non-human animal suffering from gynecological cancer; (b) measuring the expression level of the present microRNA in the gynecological cancer tissue or blood of the non-human animal; and (c) comparing the measured amount with the expression level of the microRNA in the case of the test substance being unadministered as a control for evaluation. Preferred examples of the species of the non-human animal can include a mouse, a rat, a hamster, a guinea pig, a monkey, a cow, a pig, a horse, a rabbit, sheep, a goat, a cat, and a dog; among others, a mouse and a rat can be preferably exemplified. The non-human animal suffering from gynecological cancer may be a non-human animal having spontaneously suffered from gynecological cancer or a non-human animal in which gynecological cancer has been induced using a carcinogenic substance.

The method for measuring the expression level of the present microRNA in the step (b) may use the above-described method for measuring the expression level of the present microRNA. In the comparison/evaluation method in the step (c), the test substance can be determined as having a high possibility of being a therapeutic agent for gynecological cancer when miR-592, miR-629*, miR-517a, miR-205, miR-184, miR-509-5p, miR-135a*, miR-137, miR-602, miR-186*, miR-181a-2*, miR-193b*, miR-138, miR-203, miR-488, miR-489, miR-643, miR-885-5p, or miR-888 decreases compared to a control; the test substance can be determined as having a high possibility of being a therapeutic agent for gynecological cancer when miR-377*, miR-449b, miR-449a, miR-369-3p, miR-323-3p, miR-329, miR-299-5p, miR-34b, miR-411, miR-34c-5p, miR-376b, miR-337-3p, miR-337-5p, miR-127-3p, miR-424, miR-432, miR-433, miR-450a, miR-503, miR-542-3p, miR-542-5p, or miR-584 increases compared to a control. A microRNA is reported to have properties such as binding to mRNA to suppress the expression of gene; thus, such a test substance to shift the abnormal expression of the microRNA in the gynecological cancer tissue toward normalization probably has a high possibility of being used as a therapeutic agent for gynecological cancer.

In place of the step (c), the step of (d) comparing the measured amount with the expression level of the microRNA in a non-cancer tissue of the same type as that of the gynecological cancer tissue or blood of a gynecological subject as a control for evaluation may be adopted. In the comparison/evaluation method of the step (d), the test substance can be determined as having a high possibility of being a therapeutic agent for gynecological cancer when the expression level of the present microRNA is not significantly different compared to a control.

Among the present microRNAs, miR-137, miR-138, miR-203, miR-205, miR-488, miR-489, miR-643, miR-885-5p, and miR-888 have average expression levels in cancer tissue of 5 times or more of those in non-cancer tissue, showing a large difference in the amount of expression therebetween; thus, they are preferable in that they enable the more accurate determination of a possibility that the substance is a therapeutic agent for gynecological cancer. The combined use of two or more of the above-described microRNAs as biomarkers for gynecological cancer is preferable in that it enables the more accurate determination of a possibility that the substance is a therapeutic agent for gynecological cancer.

The present invention will be more specifically described below with reference to Examples. However, these Examples are not intended to limit the technical scope of the present invention.

### Example 1

[Collection of Sample Tissue-1]

Under the approval of the ethics committee of Keio University School of Medicine, patients visiting the gynecology clinic of Keio University Hospital were selected and used as persons to be asked for sample donation. Then, after obtaining informed consent from the persons, 4 sample tissues (“SAMPLE IDS 1 to 4” in FIG. 1) were collected from 4 of the persons. The tissue types of these 4 sample tissues (the article “TISSUES” in FIG. 1) consisted of two types: endometrial polyp and endometrial cancer; and the tissue conditions (the article “SAMPLES” in FIG. 1) consisted of 2 conditions: polyp and cancer conditions.

A portion of each of the collected tissues was placed in a tube into which RNAlater (trade name) (from Applied Biosystems) was dispensed and subjected to frozen storage to stabilize RNA in each tissue. The condition (cancer, polyp, or the like) of each sample tissue was subjected to definite diagnosis by performing the pathological examination of another portion of each of the collected tissues.

### Example 2

[Extraction of RNA from Sample Tissue and Qualitative Evaluation Thereof-1]

RNA was extracted from each tissue frozen in Example 1 in order to use in a microarray to be described later. Specifically, total RNA comprising microRNAs was extracted from each of the above tissues using mirVana miRNA Isolation Kit (from Applied Biosystems) according to the appended protocol.

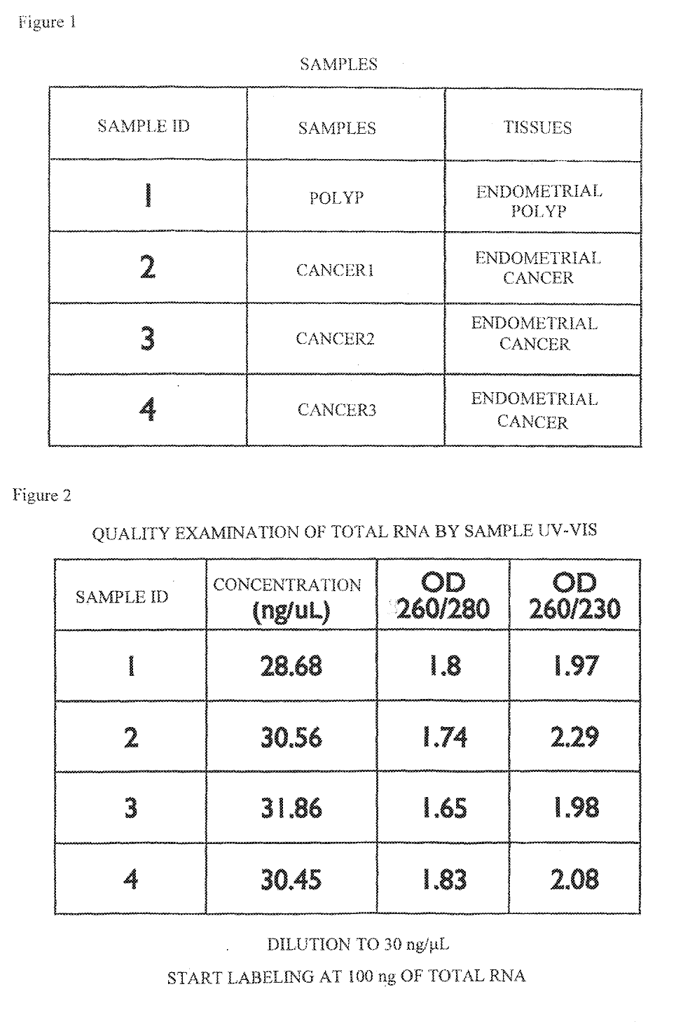

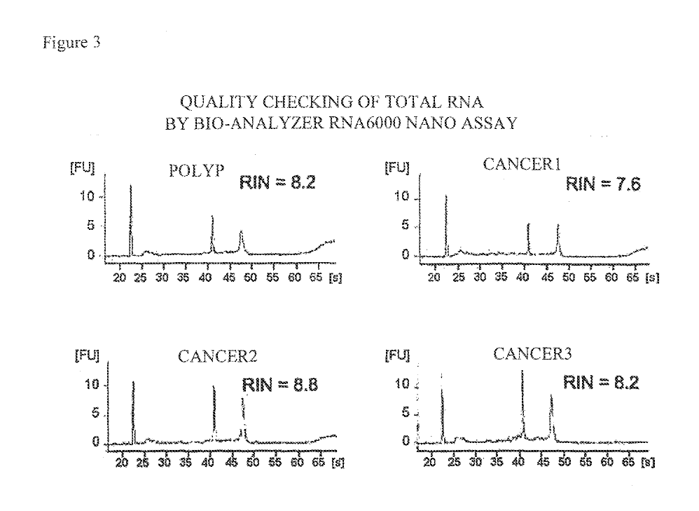

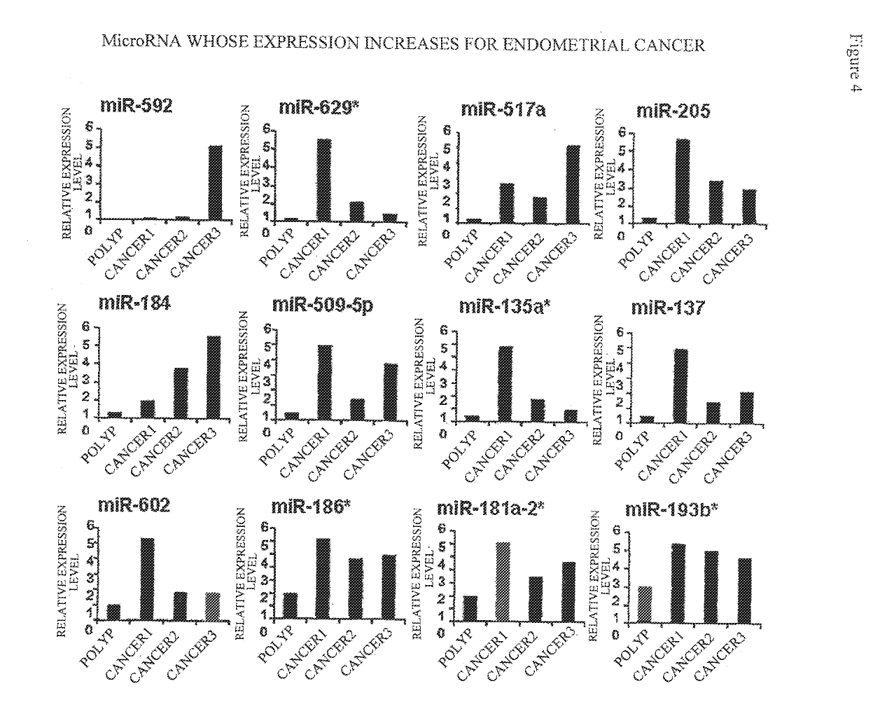

Then, the extracted RNA was subjected to qualitative evaluation in order to make sure that sufficient accuracy would be obtained in the microarray to be described later. Specifically, the resultant RNA was adjusted to a concentration of about 30 ng/μL using distilled water, and OD260/280 (the numerical value obtained by dividing the measured value of OD260 by the measured value of OD280) and the like were measured using a spectrophotometer for calculation. The results are shown in FIG. 2. As shown in FIG. 2, each OD260/280 fell in the range of about 1.6 to 2.0, indicating that each RNA was little contaminated with protein and phenol and had good quality. RIN(RNA Integrity Number) of the extracted total RNA was measured using Bio-Analyzer RNA6000 (from Agilent Technologies) to examine the degree of RNA decomposition. The results are shown in FIG. 3. As shown in FIG. 3, RIN for each sample retained a sufficient high level, indicating that each RNA had good quality.

### Example 3

[Expression Analysis of microRNAs in Each Tissue-1]

Using Agilent Human miRNA V2 (from Agilent Technologies), 723 human microRNAs were subjected to exhaustive analysis. The above-described Agilent Human miRNA V2 microarray comprises DNA sequences complementary to nucleotide sequences represented by SEQ ID NOS: 1 to 23 and 25 to 27 as probes for miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-337-3p, miR-337-5p, and miR-127-3p, respectively. The method for analysis was according to the method described in Agilent Technologies' miRNA Microarray Protocol Version 1.5. Specifically, the analysis was performed by the following method. The amount of a reagent is described as an amount for one sample.

First, the total RNA obtained in Example 2 was diluted to about 25 ng/μL with DNase/RNase-free water, and the RNA concentration of the diluted solution was measured. Then, 0.7 μL of 10×CIP Buffer (from GE Healthcare) was mixed with 0.7 μL of 16 U/4 CIP (from GE Healthcare) to prepare 1.4 μL of a CIP master mix. The above diluted solution of RNA was dispensed in such an amount that the total RNA amount is 100 ng, into a 1.5-mL tube, to which 1.4 μL of the above CIP master mix was added, followed by further adjusting the resultant solution to 7 μL with DNase/RNase-free water. Thereafter, the solution was incubated at 37° C. for 30 minutes to perform the dephosphorylation of the total RNA.

DMSO (from Sigma) (5 μL) was added to the sample solution subjected to dephosphorylation, which was then mixed and incubated at 100° C. for about 5 minutes to perform the thermal denaturation of the total RNA. Immediately thereafter, it was cooled with ice water for 2 minutes.

Then, ligation was carried out. Specifically, 2 μL of 10× T4RNA Ligase Buffer (from GE Healthcare), 2 μL of 0.1% BSA (from GE Healthcare), 1 μL of T4 RNA Ligase (from GE Healthcare) adjusted to 15 U/μL, and 3 μL of Cyanine 3-Cytidine (from Agilent Technologies) were mixed in another 1.5-mL tube to prepare a ligation master mix. The ligation master mix (8 μL) was added to the above sample subjected to thermal denaturation and then cooled, which was then incubated at 16° C. for 2 minutes.

Subsequently, the sample was purified. Specifically, using MicroBioSpin6 (from BioRad) according to the appended protocol, a sample RNA fluorescently labeled with Cyanine 3-Cytidine was purified from the above sample.

The Agilent Human miRNA V2 (from Agilent Technologies) oligo DNA microarray was used to perform the microRNA profile analysis of the above sample RNA. According to Agilent Technologies' protocol, the RNA sample was subjected to hybridization reaction at 55° C. for 20 hours or more. Next, the microarray after hybridization was washed with washing buffer 1 at room temperature for 5 minutes and with washing buffer 2 at 37° C. for 5 minutes according to the Agilent Technologies' protocol. The microarray was pulled up from the washing buffer 2 and scanned using DNA Microarray Scanner (from Agilent Technologies).

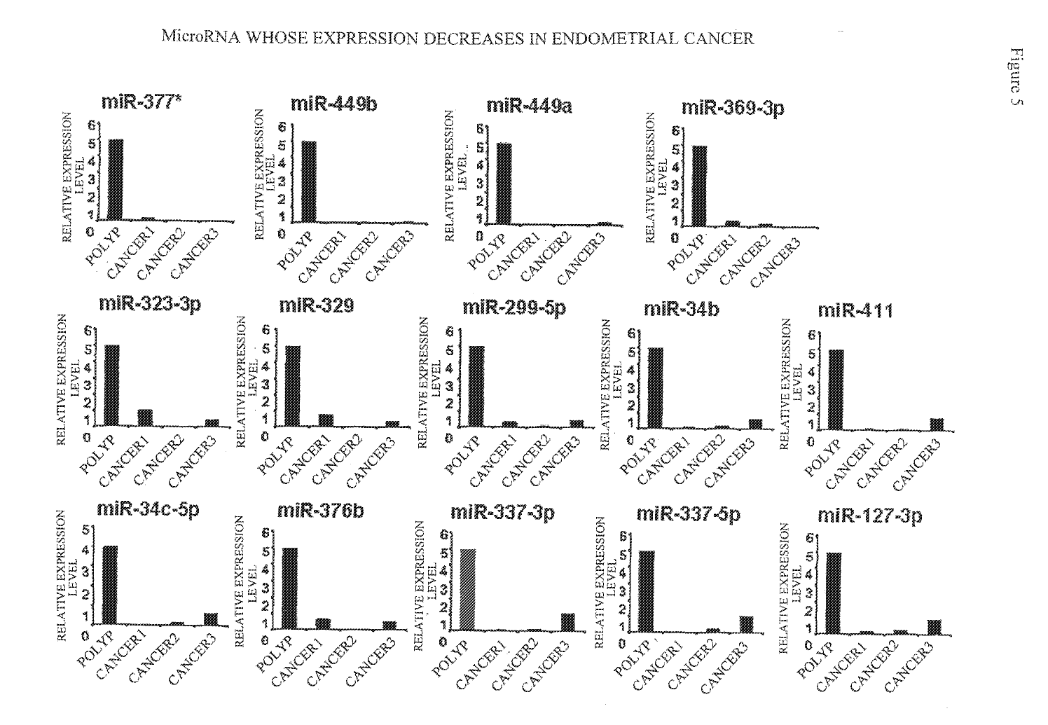

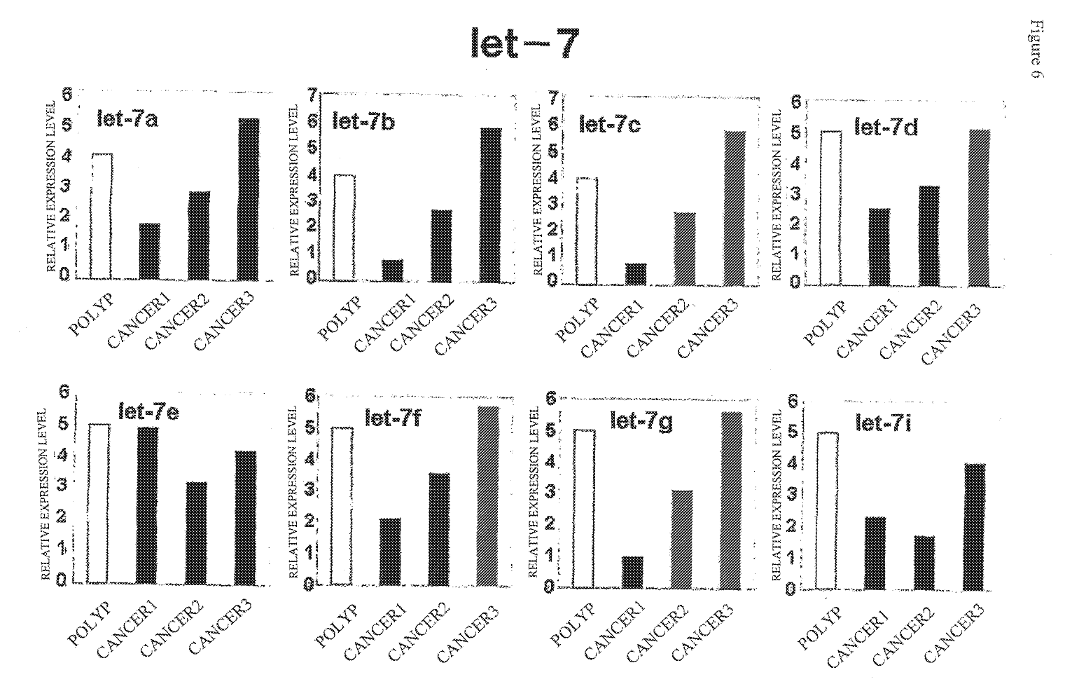

Of 723 microRNAs, a plurality of microRNAs were found which show large differences in the expression level (signal intensity) between the endometrial non-cancer tissue (POLYP) and the endometrial cancer tissue (CANCER 1 to 3). The signal intensities of these microRNAs in each sample are shown in FIGS. 4 and 5. FIG. 4 shows microRNAs whose expression increases in endometrial cancer tissues (miR-592, miR-629*, miR-517a, miR-205, miR-184, miR-509-5p, miR-135a*, miR-137, miR-602, miR-186*, miR-181a-2*, and miR-193b*); FIG. 5 shows microRNAs whose expression decreases in endometrial cancer tissues (miR-377*, miR-449b, miR-449a, miR-369-3p, miR-323-3p, miR-329, miR-299-5p, miR-34b, miR-411, miR-34c-5p, miR-376b, miR-337-3p, miR-337-5p, and miR-127-3p). These microRNAs can be used as markers for endometrial cancer tissue because the microRNAs were expressed in the endometrial cancer tissues in a manner different from the expression of those in the non-cancer tissue (abnormal expression).

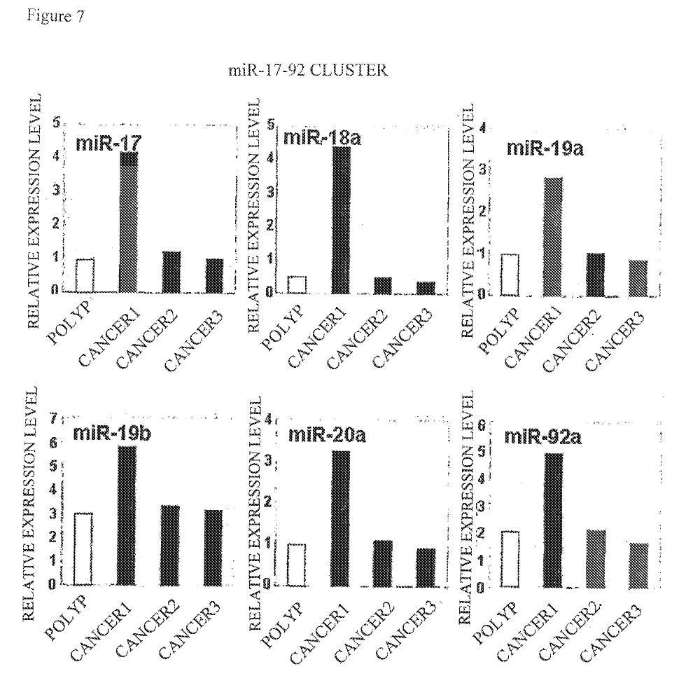

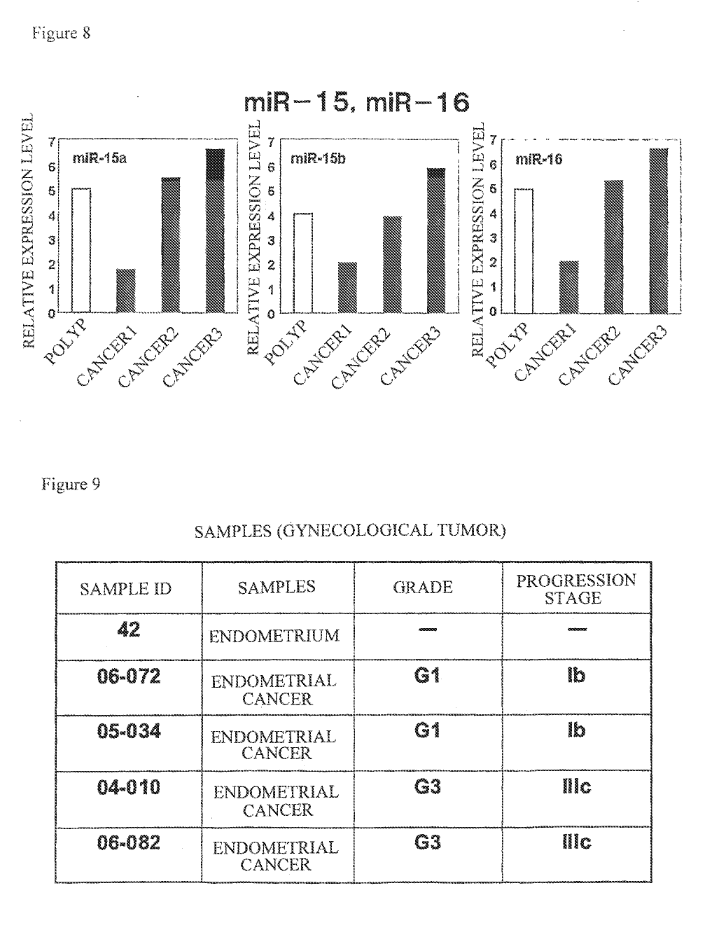

The expression of the members of the let-7 family (let-7a, let-7b, let-7c, let-7d, let-7e, let-7f, let-7g, and let-71), miR-17-92 cluster (miR-17, miR-18a, miR-19a, miR-19b, miR-20a, and miR-92a), miR-15 (miR-15a and miR-15b), and miR-16, which were known to be abnormally expressed in other cancers, was checked for each sample; however, abnormal expression was not particularly found for endometrial cancer (FIGS. 6, 7, and 8).

### Example 4

[Collection of Sample Tissue-2]

Under the approval of the ethics committee of Keio University School of Medicine, patients visiting the gynecology clinic of Keio University Hospital were selected and used as persons to be asked for sample donation. Then, after obtaining informed consent from the persons, 5 sample tissues (SAMPLE IDS “42”, “06-072”, “05-034”, “04-010”, and “06-082” in FIG. 9) were collected from 5 of the persons. The tissue types of these 5 sample tissues (the article “SAMPLES” in FIG. 9) consisted of two types: endometrium and endometrial cancer; the grades of the cancers (the article “GRADE” in FIG. 9) consisted of 2 types: G1 and G3; and the progression stage of the cancers (the article “PROGRESSION STAGE” in FIG. 9) consisted of 2 types: Ib and IIIc.

A portion of each of the collected tissues was placed in a tube into which RNAlater (trade name) (from Applied Biosystems) was dispensed and subjected to frozen storage to stabilize RNA in each tissue. The condition (cancer or the like) of each sample tissue was subjected to definite diagnosis by performing the pathological examination of another portion of each of the collected tissues.

### Example 5

[Extraction of RNA from Sample Tissue and Qualitative Evaluation Thereof-2]

RNA was extracted from each tissue frozen in Example 4 in order to use for real-time PCR to be described later. Specifically, total RNA comprising microRNAs was extracted from each of the above tissues using mirVana miRNA Isolation Kit (from Applied Biosystems) according to the appended protocol.

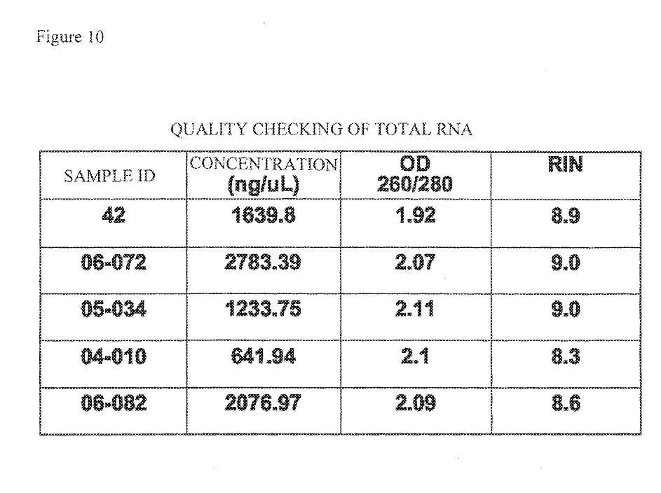

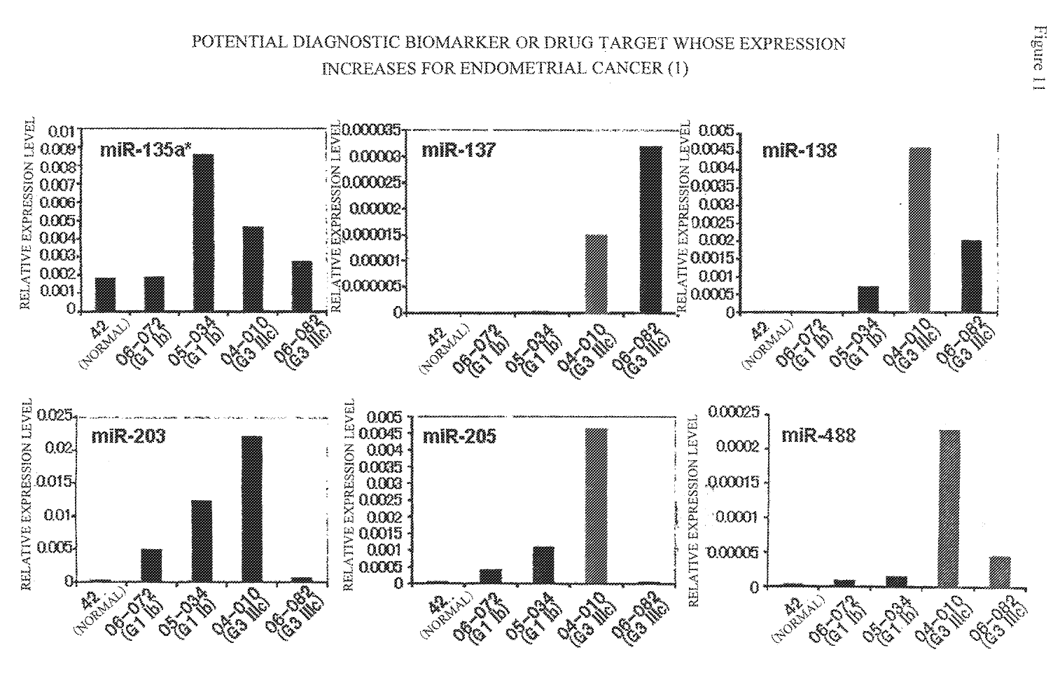

Then, the extracted RNA was subjected to qualitative evaluation in order to make sure that sufficient accuracy would be obtained in the real-time PCR to be described later. Specifically, the resultant RNA was adjusted to a concentration of about 30 ng/μL using distilled water, and OD260/280 (the numerical value obtained by dividing the measured value of OD260 by the measured value of OD280) and the like were measured and calculated using a spectrophotometer. The results are shown in FIG. 10. As shown in FIG. 10, each OD260/280 fell in the range of about 1.6 to 2.0, indicating that each RNA was little contaminated with protein and phenol and had good quality. RIN(RNA Integrity Number) of the extracted total RNA was measured using Bio-Analyzer RNA6000 (from Agilent Technologies) to examine the degree of RNA decomposition. The results are shown in FIG. 10. As shown in FIG. 10, RIN for each sample retained a sufficient high level, indicating that each RNA had good quality.

### Example 6

[Expression Analysis of microRNAs in Each Tissue-2]

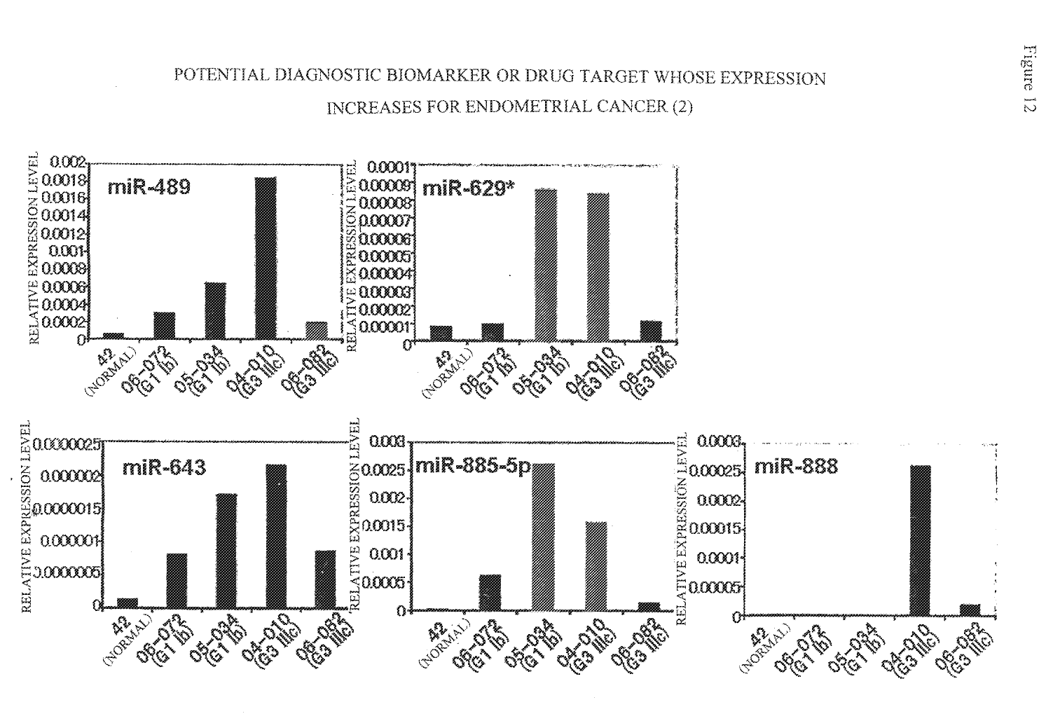

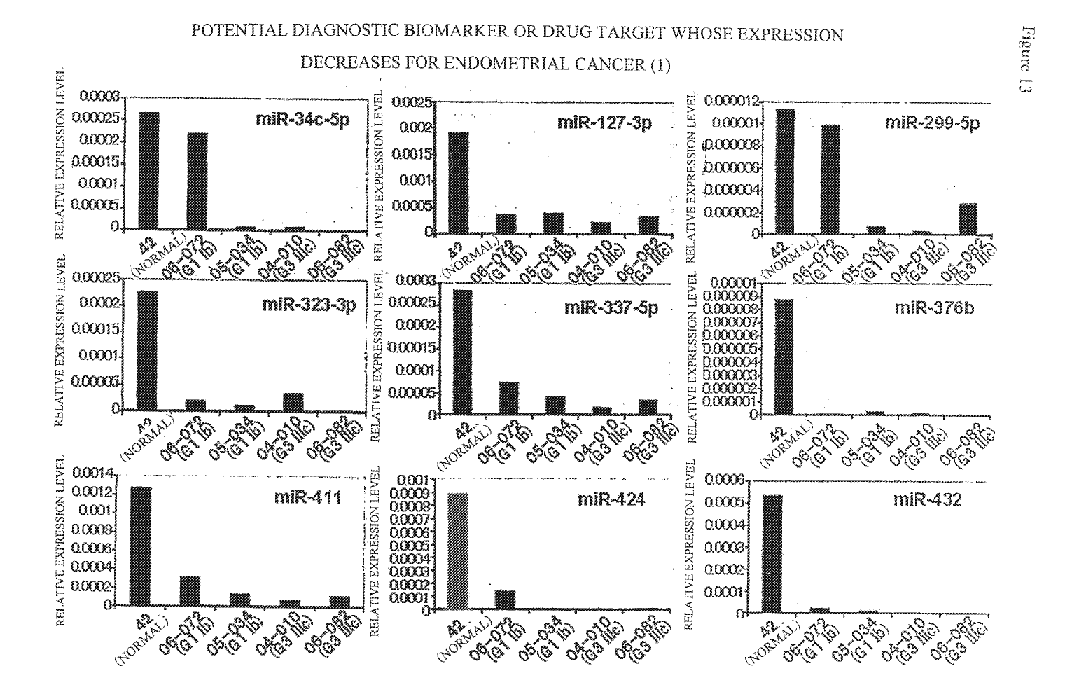

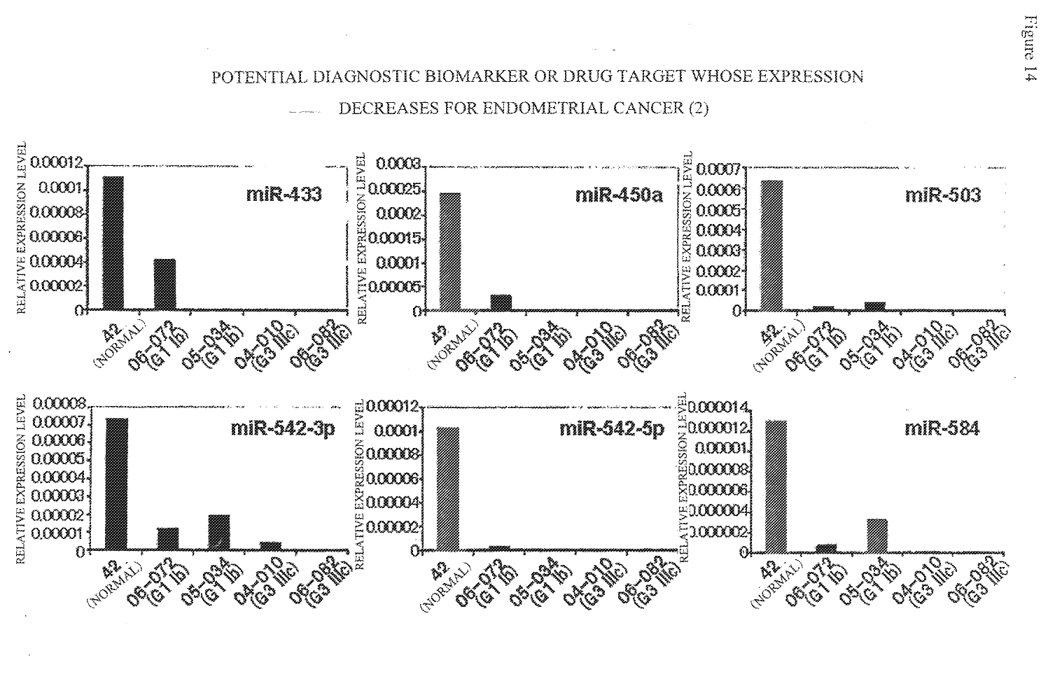

Using the RNA extracted in the above Example 5 and TaqMan (trade name) MicroRNA Assays (from Applied Biosystems), human microRNAs were subjected to exhaustive analysis by the real-time PCR method. The method for quantitative determination was according to the method described in the appended protocol. In each sample, the results of quantitatively determining miR-135a*, miR-137, miR-138, miR-203, miR-205, and miR-488 are shown in FIG. 11; the results of quantitatively determining miR-489, miR-629*, miR-643, miR-885-5p, and miR-888, in FIG. 12; the results of quantitatively determining miR-34c-5p, miR-127-3p, miR-299-5p, miR-323-3p, miR-337-5p, miR-376b, miR-411, miR-424, and miR-432, in FIG. 13; and the results of quantitatively determining miR-433, miR-450a, miR-503, miR-542-3p, miR-542-5p and miR-584, in FIG. 14. As shown in FIGS. 11 to 12, the expression of miR-135a*, miR-137, miR-138, miR-203, miR-205, miR-488, miR-489, miR-629*, miR-643, miR-885-5p, and miR-888 could be confirmed to increase in the endometrial cancer tissues compared to that in the normal endometrial tissue (NORMAL). As shown in FIGS. 13 to 14, the expression of miR-34c-5p, miR-127-3p, miR-299-5p, miR-323-3p, miR-337-5p, miR-376b, miR-411, miR-424, miR-432, miR-433, miR-450a, miR-503, miR-542-3p, miR-542-5p and miR-584 could be confirmed to decrease in the endometrial cancer tissues compared to that in the normal endometrial tissue (NORMAL).

1. A method for using one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 as a biomarker for gynecological cancer.

2. The method according to claim 1, wherein the miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 are RNAs in which one or more nucleotides are deleted, substituted or added in the nucleotide sequences represented by SEQ ID NOS: 1 to 27 and 50 to 63, respectively and whose expression increases or decreases in a gynecological cancer tissue or blood of a gynecological cancer subject compared to a control.

3. A method for the determination of gynecological cancer, comprising the steps of:
(A) extracting RNA from a gynecological specimen tissue or blood of a subject;
(B) measuring the expression level of one or more microRNAs selected from miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 in the extracted RNA; and
(C) comparing the measured expression level to the expression level of the microRNAs in a non-cancer tissue of the same type as that of the specimen tissue or in blood of a normal subject of the same species as that of the above subject as a control for evaluation.

4. The method for the determination of gynecological cancer according to claim 3, wherein the miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 are RNAs in which one or more nucleotides are deleted, substituted or added in the nucleotide sequences represented by SEQ ID NOS: 1 to 27 and 50 to 63, respectively and whose expression increases or decreases in the gynecological cancer tissue or the blood of the gynecological cancer subject compared to a control.

5. The method for the determination of gynecological cancer according to claim 3 or 4, wherein the cancer is determined as being gynecological cancer when the expression level of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-138, miR-203, miR-488, miR-489, miR-643, miR-885-5p, and miR-888 in the extracted RNA increases compared to a control.

6. The method for the determination of gynecological cancer according to claim 3 or 4, wherein the cancer is determined as being gynecological cancer when the expression level of one or more microRNAs selected from the group of microRNAs consisting of miR-377*, miR-449b, miR-449a, miR-369-3p, miR-323-3p, miR-329, miR-299-5p, miR-34b, miR-411, miR-34c-5p, miR-376b, miR-337-3p, miR-337-5p, miR-127-3p, miR-424, miR-432, miR-433, miR-450a, miR-503, miR-542-3p, miR-542-5p and miR-584 in the extracted RNA decreases compared to a control.

7. A kit for the determination of gynecological cancer, comprising a microarray which includes a polynucleotide consisting of a nucleic acid sequence complementary to the sequence of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 or a part thereof, and which is capable of measuring the expression level of the microRNA.

8. A kit for the determination of gynecological cancer, including a primer set capable of amplifying the sequence of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 and a fluorescent probe comprising a polynucleotide consisting of a nucleic acid sequence complementary to the microRNA sequence or a part thereof.

9. The determination kit according to claim 7 or 8, wherein the miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 are RNAs in which one or more nucleotides are deleted, substituted or added in the nucleotide sequences represented by SEQ ID NOS: 1 to 27 and 50 to 63, respectively and whose expression increases or decreases in a gynecological cancer tissue or blood of gynecological cancer subjects compared to a control.

10. A method for screening a therapeutic agent for gynecological cancer, comprising the steps of:
(A) administering a test substance to a non-human animal suffering from gynecological cancer;
(B) measuring the expression level of one or more microRNAs selected from the group of microRNAs consisting of miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 in a gynecological cancer tissue or blood of the non-human animal; and
(C) comparing the above expression level to the expression level of the microRNA in the case of the test substance being unadministered as a control for evaluation.

11. The screening method according to claim 10, wherein the miR-592, miR-629*, miR-517a, miR-205, miR-184, miR-509-5p, miR-135a*, miR-137, miR-602, miR-186*, miR-181a-2*, miR-193b*, miR-377*, miR-449b, miR-449a, miR-369-3p, miR-323-3p, miR-329, miR-299-5p, miR-34b, miR-411, miR-34c-5p, miR-376b, miR-885-5p, miR-337-3p, miR-337-5p, miR-127-3p, miR-138, miR-203, miR-488, miR-489, miR-643, miR-888, miR-424, miR-432, miR-433, miR-450a, miR-503, miR-542-3p, miR-542-5p and miR-584 are RNAs in which one or more nucleotides are deleted, substituted or added in the nucleotide sequences represented by SEQ ID NOS: 1 to 27 and 50 to 63, respectively and whose expression increases or decreases in the gynecological cancer tissue or the blood of the gynecological cancer subject compared to a control.


SEQUENCE LISTING (63 sequences)

SEQ ID NO: 1 - RNA, 22 nt, Homo sapiens
uugugucaauaugcgaugaugu

SEQ ID NO: 2 - RNA, 22 nt, Homo sapiens
guucucccaacguaagcccagc

SEQ ID NO: 3 - RNA, 23 nt, Homo sapiens
aucgugcaucccuuuagaguguu

SEQ ID NO: 4 - RNA, 22 nt, Homo sapiens
uccuucauuccaccggagucug

SEQ ID NO: 5 - RNA, 22 nt, Homo sapiens
uggacggagaacugauaagggu

SEQ ID NO: 6 - RNA, 21 nt, Homo sapiens
uacugcagacaguggcaauca

SEQ ID NO: 7 - RNA, 22 nt, Homo sapiens
uauagggauuggagccguggcg

SEQ ID NO: 8 - RNA, 22 nt, Homo sapiens
uauugcuuaagaauacgcguag

SEQ ID NO: 9 - RNA, 23 nt, Homo sapiens
gacacgggcgacagcugcggccc

SEQ ID NO: 10 - RNA, 22 nt, Homo sapiens
gcccaaaggugaauuuuuuggg

SEQ ID NO: 11 - RNA, 22 nt, Homo sapiens
accacugaccguugacuguacc

SEQ ID NO: 12 - RNA, 22 nt, Homo sapiens
cgggguuuugagggcgagauga

SEQ ID NO: 13 - RNA, 22 nt, Homo sapiens
agagguugcccuuggugaauuc

SEQ ID NO: 14 - RNA, 22 nt, Homo sapiens
aggcaguguauuguuagcuggc

SEQ ID NO: 15 - RNA, 22 nt, Homo sapiens
uggcaguguauuguuagcuggu

SEQ ID NO: 16 - RNA, 21 nt, Homo sapiens
aauaauacaugguugaucuuu

SEQ ID NO: 17 - RNA, 21 nt, Homo sapiens
cacauuacacggucgaccucu

SEQ ID NO: 18 - RNA, 22 nt, Homo sapiens
aacacaccugguuaaccucuuu

SEQ ID NO: 19 - RNA, 22 nt, Homo sapiens
ugguuuaccgucccacauacau

SEQ ID NO: 20 - RNA, 23 nt, Homo sapiens
uaggcagugucauuagcugauug

SEQ ID NO: 21 - RNA, 21 nt, Homo sapiens
uaguagaccguauagcguacg

SEQ ID NO: 22 - RNA, 23 nt, Homo sapiens
aggcaguguaguuagcugauugc

SEQ ID NO: 23 - RNA, 22 nt, Homo sapiens
aucauagaggaaaauccauguu

SEQ ID NO: 24 - RNA, 22 nt, Homo sapiens
uccauuacacuacccugccucu

SEQ ID NO: 25 - RNA, 22 nt, Homo sapiens
cuccuauaugaugccuuucuuc

SEQ ID NO: 26 - RNA, 21 nt, Homo sapiens
gaacggcuucauacaggaguu

SEQ ID NO: 27 - RNA, 22 nt, Homo sapiens
ucggauccgucugagcuuggcu

SEQ ID NO: 28 - RNA, 22 nt, Homo sapiens
ugagguaguagguuguauaguu

SEQ ID NO: 29 - RNA, 22 nt, Homo sapiens
ugagguaguagguugugugguu

SEQ ID NO: 30 - RNA, 22 nt, Homo sapiens
ugagguaguagguuguaugguu

SEQ ID NO: 31 - RNA, 21 nt, Homo sapiens
agagguaguagguugcauagu

SEQ ID NO: 32 - RNA, 21 nt, Homo sapiens
ugagguaggagguuguauagu

SEQ ID NO: 33 - RNA, 22 nt, Homo sapiens
ugagguaguagauuguauaguu

SEQ ID NO: 34 - RNA, 21 nt, Homo sapiens
ugagguaguaguuuguacagu

SEQ ID NO: 35 - RNA, 21 nt, Homo sapiens
ugagguaguaguuugugcugu

SEQ ID NO: 36 - RNA, 23 nt, Homo sapiens
caaagugcuuacagugcagguag

SEQ ID NO: 37 - RNA, 22 nt, Homo sapiens
uaaggugcaucuagugcagaua

SEQ ID NO: 38 - RNA, 23 nt, Homo sapiens
ugugcaaaucuaugcaaaacuga

SEQ ID NO: 39 - RNA, 23 nt, Homo sapiens
ugugcaaauccaugcaaaacuga

SEQ ID NO: 40 - RNA, 23 nt, Homo sapiens
uaaagugcuuauagugcagguag

SEQ ID NO: 41 - RNA, 22 nt, Homo sapiens
uauugcacuugucccggccugu

SEQ ID NO: 42 - RNA, 22 nt, Homo sapiens
uagcagcacauaaugguuugug

SEQ ID NO: 43 - RNA, 22 nt, Homo sapiens
uagcagcacaucaugguuuaca

SEQ ID NO: 44 - RNA, 22 nt, Homo sapiens
uagcagcacguaaauauuggcg

SEQ ID NO: 45 - RNA, 23 nt, Mus musculus
auugugucaauaugcgaugaugu

SEQ ID NO: 46 - RNA, 21 nt, Mus musculus
aggcagugcauugcuagcugg

SEQ ID NO: 47 - RNA, 20 nt, Mus musculus
aauaauacaugguugaucuu

SEQ ID NO: 48 - RNA, 22 nt, Mus musculus
aacacacccagcuaaccuuuuu

SEQ ID NO: 49 - RNA, 23 nt, Mus musculus
aacacgguccacuaacccucagu

SEQ ID NO: 50 - RNA, 23 nt, Homo sapiens
agcugguguugugaaucaggccg

SEQ ID NO: 51 - RNA, 22 nt, Homo sapiens
gugaaauguuuaggaccacuag

SEQ ID NO: 52 - RNA, 21 nt, Homo sapiens
uugaaaggcuauuucuugguc

SEQ ID NO: 53 - RNA, 22 nt, Homo sapiens
gugacaucacauauacggcagc

SEQ ID NO: 54 - RNA, 22 nt, Homo sapiens
acuuguaugcuagcucagguag

SEQ ID NO: 55 - RNA, 21 nt, Homo sapiens
uacucaaaaagcugucaguca

SEQ ID NO: 56 - RNA, 22 nt, Homo sapiens
cagcagcaauucauguuuugaa

SEQ ID NO: 57 - RNA, 23 nt, Homo sapiens
ucuuggaguaggucauugggugg

SEQ ID NO: 58 - RNA, 22 nt, Homo sapiens
aucaugaugggcuccucggugu

SEQ ID NO: 59 - RNA, 22 nt, Homo sapiens
uuuugcgauguguuccuaauau

SEQ ID NO: 60 - RNA, 23 nt, Homo sapiens
uagcagcgggaacaguucugcag

SEQ ID NO: 61 - RNA, 22 nt, Homo sapiens
ugugacagauugauaacugaaa

SEQ ID NO: 62 - RNA, 23 nt, Homo sapiens
ucggggaucaucaugucacgaga

SEQ ID NO: 63 - RNA, 22 nt, Homo sapiens
uuaugguuugccugggacugag